(19) United States
(12) Patent Application Publication
(10) Pub. No.: US 2002/0099274 A1
(43) Pub. Date: Jul. 25, 2002

(54) Portable self-administered health care terminal and a self-administered health support system

(75) Inventors: Eiji Isomura, Tokyo (JP); Yasutane Takafuji, Yokohama-shi (JP)

Correspondence Address: LOWE HAUPTMAN GOPSTEIN GILMAN & BERNER, LLP, Suite 310, 1700 Diagonal Road, Alexandria, VA, 22314, US

(73) Assignee: I.S.C.G. CO., LTD

(21) Appl. No.: 09/920,010

(22) Filed: Aug. 2, 2001

(30) Foreign Application Priority Data
Jan. 24, 2001 (JP) 2001-016255

(51) Int. Cl. A61B 5/00

(52) U.S. Cl. 600/300

(57) ABSTRACT

A patient carrying a portable self-administered health care terminal receives data regarding the energy value per nutrient to be consumed daily and the amount of energy to expend through exercise. When dining at a restaurant or other facility providing food, the patient uses a barcode reader connected to the portable terminal to read barcodes included in the menu. This barcode data is transmitted to a server in the facility. The server returns data regarding the energy value per nutrient in the selected foods. The data is automatically input into the portable terminal.

### BACKGROUND OF THE INVENTION

1. Field of the Invention

The present invention relates to a portable self-administered health care terminal and a self-administered health support system for the effective management of food intake, exercise, and the like. The users of this health care terminal and system include patients following treatment for lifestyle-related diseases such as diabetes that includes therapeutic exercise and diet regimens, people deemed highly susceptible to adult diseases and require management of their everyday lifestyle, and people who wish to maintain health and follow a healthy diet plan.

2. Description of the Related Art

Conventionally, therapeutic exercise and diet regimens have been prescribed in medical institutions as treatment for diabetes and other lifestyle-related diseases based on the condition of the patient. For therapeutic exercises, the patient jogs, swims, or performs activities using an auxiliary device such as a treadmill to expend an amount of energy prescribed by the medical institution. The patients themselves record by hand data regarding the amount of energy expended.

In the dietetic therapy, patients are instructed to consume foods that meet the nutrient energy values (calories) prescribed by their medical institution. However, when dining at restaurants, the patient must guess at the types and weights of ingredients used in the cooked foods. Then, using a food composition table that lists the nutrient energy per unit weight for various ingredients, the user calculates the nutrient energy in the prepared foods based on their guesses and records the data by hand. When buying foods at convenience stores or the like, the patient reads the nutrient composition listed on the processed foods, calculates the nutrient energy for each ingredient, and records the values by hand.

It is necessary for medical institutions to understand the patient's biological data (blood pressure, blood sugar, etc.), the amount of energy expended in exercise, and the amount of food consumed in order to prescribe an exercise routine and diet regimen to treat the patient's lifestyle-related disease. It is particularly desirable to gain an accurate understanding of the amounts of energy and nutrients taken in daily.

Accordingly, patients have conventionally performed exercises such as jogging, swimming, or other exercises using auxiliary equipment, such as a treadmill, to expend an amount of energy prescribed by the medical institutions. These same patients have recorded the amounts of expended energy by hand.

For the diet regiment, patients themselves ordinarily estimate types and weights of ingredients when dining out or the like. The patients then calculate the amounts of energy and nutrients included in the food based on a food composition table that lists this data based on units of weight and record the data by hand.

When consuming foods purchased at convenience stores or the like, the patient calculates the nutrient energy for ingredients based on a composition table listed on the processed food packages and records this data by hand.

The process of recording by hand all the energy amounts expended through exercise and calculated values of nutrient energy in ingested foods is a troublesome task and places an extremely great burden on the patient, both physically and mentally. Further, since some calculations are based on estimates and the data is being written by hand, there is a large potential for inaccuracies in the data. For these reasons, some patients do not continue to maintain records of this data for an extended period of time and often the effects of the treatment are not realized.

Often therapeutic exercise and diet regimens are continued over a long period of time. Since patients who undergo such treatment have a great burden, they sometimes feel uneasy during the treatment or become emotionally depressed. It has been suggested that counseling and consultation institutes are needed to alleviate the uneasiness and emotional burden of the patients, but not enough has been done to meet this need.

Further, the public has voiced a request for the provision of a system capable of effectively supporting treatment in order to restrain outbreaks and progression of rapidly increasing lifestyle diseases and to reduce medical fees that are continually rising.

### SUMMARY OF THE INVENTION

In view of the foregoing, it is an object of the present invention to provide a portable self-administered health care terminal and a self-administered health support system to effectively support therapeutic exercise and diet regimens in order to lighten the burden on the patients.

These objects and others will be attained by a portable self-administered health care terminal for supporting self-management of diet and exercise, comprising communication means for exchanging data with external equipment via a communications network; display means for receiving and displaying data regarding recommended amounts of energy per nutrient to be consumed, daily required exercise, and other data required for treatment from a server at a health care facility for supporting treatment for users with portable terminals; input means for inputting data concerning the energy contained in consumed foods and energy expended through exercise; reception means for receiving data regarding amounts of nutrient energy contained in foods about to be consumed from a server at a food provision facility equipped with a database that stores amounts of nutrient energy contained in foods that the facility provides; and transmission means for transmitting data regarding energy contained in consumed foods and energy expended by exercise to the server at the health care facility.

According to another aspect of the present invention, the portable self-administered health care terminal comprises communication means for exchanging data with external equipment via a communications network; display means for receiving and displaying data regarding amounts of daily required exercise, nutrient energy that should be consumed, instructions on dosage and administration of medicine, instructions on taking biological measurements, and other data required for treatment from a server at a health care facility for supporting treatment for users with portable terminals; input means for inputting with key operations data regarding the nutrient energy in consumed foods; input means for inputting with key operations data regarding the energy expended in exercise; transmission means for reading patterns or numbers from a restaurant menu and transmitting that data to a server of a food provision facility in order to receive data regarding amounts of nutrient energy contained in foods about to be consumed from a server at a food provision facility equipped with a database that stores amounts of nutrient energy contained in foods that the facility provides; reception means for receiving data regarding amounts of nutrient energy contained in foods about to be consumed from the server at the food provision facility; input means for inputting data from a motion measuring device for detecting movement of a human body during exercise and measuring the amount of energy expended; input means for inputting data from a sphygmomanometer and other biological data measuring devices; prompting means for prompting the patient to take medicine and perform biological measurements; display means for displaying data for the amount of energy consumed through food and data for the amount of energy expended through exercise; and transmission means for transmitting data regarding energy consumed through foods, energy expended through exercise, instructions on dosage and administration of medicine and instructions on taking biological measurements to the server at the health care facility.

According to another aspect of the present invention, the portable self-administered health care terminal comprises communication means for exchanging data with external equipment via a communications network, wireless telephone network, and wireless telephone; display means that uses the functions of a wireless telephone to receive and display data regarding amounts of daily required exercise, nutrient energy that should be consumed, instructions on dosage and administration of medicine, instructions on taking biological measurements, and other data required for treatment from a server at a health care facility for supporting treatment for users with portable terminals; input means for inputting with key operations data regarding the nutrient energy in consumed foods; input means for inputting with key operations data regarding the energy expended in exercise; transmission means for reading patterns or numbers from a restaurant menu and transmitting that data via a wireless communication device to a server of a food provision facility in order to receive data regarding amounts of nutrient energy contained in foods about to be consumed from a server at a food provision facility equipped with a database that stores amounts of nutrient energy contained in foods that the facility provides; reception means for receiving data via a wireless communication device from the server at the food provision facility regarding amounts of nutrient energy contained in foods about to be consumed; means for displaying food consumption after determining the correctness there of based on data regarding amounts of nutrient energy contained in foods about to be consumed and the recommended daily amounts of nutrient energy received from the health care facility; input means for inputting data from a motion measuring device for detecting movement of a human body during exercise and measuring the amount of energy expended; means for recording data from a sphygmomanometer and other biological data measuring devices and data from a treadmill and other auxiliary exercise equipment onto an IC card or other portable storage medium, and for inputting data stored on the portable storage medium into the portable terminal via a storage medium reading/writing device provided in the portable terminal; prompting means for prompting the patient to take medicine and perform biological measurements; display means for displaying data for the amount of energy consumed through food and data for the amount of energy expended through exercise; and transmission means for using the functions of a wireless telephone to transmit data regarding energy consumed through foods, energy expended through exercise, instructions on dosage and administration of medicine and instructions on taking biological measurements to the server at the health care facility.

According to another aspect of the present invention, the patterns provided in the menus at the food provision facility are barcodes and a barcode reader is used to input barcode data into the portable terminal.

According to another aspect of the present invention, the portable terminal is provided with one or all of a wireless telephone, a wireless communication device for transmitting and receiving data between the food provision facility, a motion measuring device, a device for reading the patterns or numbers recorded in the menus, and a device for reading from and writing to the portable recording medium. Further, the communications network is the Internet.

According to another aspect of the present invention, a self-administered health support system for supporting a patient's self-administration of diet and exercise, comprises a server at a health care facility; a server at a food provision facility; a server at a medical institute; a portable terminal carried by the patient; and a communication network linking the above components.

The health care facility server comprises communicating means for exchanging data with external equipment via the communication network; database means for storing data regarding treatment methods for the patient and the status of treatment; receiving means for receiving data from the medical institution regarding prescribed treatment methods for patients of the institution; transmitting means for transmitting daily amounts of required exercise and energy per nutrient to be consumed, and other data required for treatment, to the portable terminal of the patient; and recording means for receiving and recording data from the portable terminal of the patient regarding amounts of energy expended through exercise, amounts of energy per nutrient consumed, and other data related to treatment.

The food provision facility server comprises communicating means for exchanging data with external equipment via the communication network; database means for storing data regarding energy per nutrient in foods provided by the food provision facility; and searching and transmitting means for searching the database means in response to requests from the portable terminal and transmitting to that terminal data regarding the energy per nutrient in the requested foods.

The medical institution server comprises communicating means for exchanging data with external equipment via the communication network; transmitting means for transmitting data regarding the treatment methods for the patient to the health care facility server; and referencing means for referencing data regarding the status of treatment for the patient recorded in a database at the health care facility.

The portable terminal comprises communicating means for exchanging data with external equipment via the communication network; receiving and displaying means for receiving and displaying daily amounts of required exercise and energy per nutrient to be consumed, and other data required for treatment, from the health care facility server; inputting means for inputting data regarding the amounts of energy in consumed foods and the amounts of energy expended through exercise; receiving means for receiving data from the food provision facility server regarding the energy per nutrient in foods about to be consumed at the food provision facility; and transmitting means for transmitting data regarding the amounts of energy in consumed foods, the amounts of energy expended through exercise, and other data related to treatment, to the health care facility server.

According to another aspect of the present invention, the health care facility server comprises communicating means for exchanging data with external equipment via the communication network; database means for storing personal data on the patient, data regarding treatment methods for the patient, data regarding the status of therapeutic exercise and diet regimens followed by the patient, data on medicines taken by the patient, and biological measurements of the patient; receiving and storing means for receiving and storing data from the medical institution including personal data on the patient, prescribed treatment methods for the patients, and other data required for treatment; transmitting means for transmitting daily amounts of required exercise and energy per nutrient to be consumed, dosage and administration of medicines, instructions for biological measurements, and other data required for treatment, to the portable terminal of the patient; and recording means for receiving and recording data from the portable terminal of the patient regarding amounts of energy expended through exercise, amounts of energy per nutrient consumed, dosage and administration of medicines, execution of biological measurements, and other data related to treatment.

The food provision facility server comprises communicating means for exchanging data with external equipment via the communication network; database means for storing data regarding energy per nutrient in foods provided by the food provision facility; and searching and transmitting means for searching the database means in response to requests from the portable terminal and transmitting to that terminal data regarding the energy per nutrient in the requested foods.

The medical institution server comprises communicating means for exchanging data with external equipment via the communication network; transmitting means for transmitting personal data on the patient, prescribed treatment methods for the patient, and other data required for treatment to the health care facility server; and referencing means for referencing data regarding the status of therapeutic exercise and diet regimens followed by the patient, data on medicines taken by the patient, and biological measurements of the patient recorded in a database at the health care facility.

The portable terminal comprises communicating means for exchanging data with external equipment via the communication network; receiving and displaying means for receiving and displaying daily amounts of required exercise and energy per nutrient to be consumed, dosage and administration of medicines, instructions for biological measurements, and other data required for treatment from the health care facility server; inputting means for inputting data regarding the amounts of energy per nutrient in consumed foods; inputting means for inputting the amounts of energy expended through exercise; receiving means for receiving data from the food provision facility server regarding the energy per nutrient in foods about to be consumed at the food provision facility; inputting means for inputting data from a motion measuring device that detects human movement and measures the amount of energy consumed through that movement; inputting means for inputting data from a sphygmomanometer and other biological data measuring devices, and data from a treadmill and other auxiliary exercise equipment; instructing means for prompting the patient to take medicines and measure biological data; display means for displaying data regarding the amount of energy in foods consumed and the amount of energy expended through exercise; and transmitting means for transmitting data regarding the amounts of energy in consumed foods, the amounts of energy expended through exercise, and biological data measurements to the health care facility server.

According to another aspect of the present invention, the health care facility server further comprises database means for storing data regarding the energy per nutrient in various foods; and means for receiving data from the food provision facility server regarding foods provided at that facility, calculating the amounts of energy per nutrient in those foods based on data stored in the database means, and returning the calculated data to the food provision facility server.

The food provision facility server further comprises transmitting means for transmitting requests to calculate the energy per nutrients included in foods provided at the food provision facility to the server at the health care facility; and database means for receiving and storing from the health care facility server calculated data on the energy per nutrients included in foods provided at the food provision facility.

According to another aspect of the present invention, the self-administered health support system further comprises means for including names of foods and patterns or numbers to serve as an index for extracting data regarding energy per nutrient in the foods from the database in a menu of foods provided by the food provision facility in order to transmit such data from the server at the food provision facility to the patient's portable terminal; wireless communication means for exchanging data wirelessly with the portable terminal; and extracting and transmitting means for extracting data on energy per nutrient for foods stored in the database based on indexes transmitted wirelessly from the portable terminal and transmitting the extracted data to the portable terminal.

The portable terminal further comprises wireless communication means for exchanging data wirelessly with the food provision facility terminal; inputting means for inputting data from a device for reading patterns or numbers; transmitting means for transmitting index data based on the patterns or numbers read from the menu to the food provision facility server wirelessly; receiving means for receiving data regarding energy per nutrient in the foods from the food provision facility server; and display means for determining and displaying the appropriateness of consuming the foods based on data regarding energy per nutrient in the selected foods and energy per nutrient to be consumed daily, as directed by the medical institution.

According to another aspect of the present invention, the patterns provided in the menus at the food provision facility are barcodes, and the device for reading the patterns is a barcode reader. Further, a portable telephone is used as the communicating means of the portable terminal for communicating with the communication network.

Further, the food provision facility server and the portable terminal further comprise a wireless communication device as the transmitting and receiving means between the food provision facility server and the portable terminal.

Further, an IC card or other portable storage medium is used as the inputting means for inputting data from the biological data measuring device into the portable terminal, and the portable terminal further comprises a device for reading from and writing to the portable storage medium.

According to another aspect of the present invention, some or all of the device for reading patterns and numbers, the wireless communication device for communicating with the food provision facility, the motion measuring device, and the device for reading from and writing to the portable storage medium are built into the portable terminal.

According to another aspect of the present invention, a portable telephone function is built into the portable terminal and some or all of the device for reading patterns and numbers, the wireless communication device for communicating with the food provision facility, the motion measuring device, and the device for reading from and writing to the portable storage medium are built into the portable terminal.

According to another aspect of the present invention, a plurality of health care facilities are provided according to the number of users or the size of a controlling region, with the present system being managed by each health care facility, and the servers of each health care facility are interconnected via a communication network. Further, the communications network is the Internet.

According to another aspect of the present invention, the server at the health care facility is a web server comprising means for presenting a web page on the Internet; means for providing data to the user via the web page regarding guidance in using the system and treatment; means for answering questions from users via the web page; means for providing counseling to users via the web page in order to alleviate treatment concerns and emotional distress; means for exchanging opinions with other users via the web page; and means for providing via electronic mail data regarding the user's self-administered status to a health insurance institution that is engaged in a contract with the user after receiving consent from the user.

### BRIEF DESCRIPTION OF THE DRAWINGS

In the drawings:

FIG. 1 is a block diagram showing a self-administered health support system of the present invention;

FIG. 2 is a flowchart showing the early morning operations of the system;

FIG. 3 is a flowchart showing the morning operations of the system;

FIG. 4 is a flowchart showing the afternoon operations of the system;

FIG. 5 is a flowchart showing the evening operations of the system;

FIG. 6 is a table showing an example of energy per nutrient consumed during one day;

FIG. 7 includes a table and graph displayed in the portable terminal to show the energy per nutrient to be consumed in one day;

FIG. 8 includes a table and graph displayed in the portable terminal to show the energy per nutrient to be consumed for breakfast;

FIG. 9 includes a table and graph displayed in the portable terminal to show the energy per nutrient to be consumed for lunch;

FIG. 10 includes a table and graph displayed in the portable terminal to show the energy per nutrient to be consumed for dinner;

FIG. 11 includes a table and graph displayed in the portable terminal to show the energy per nutrient consumed during the day;

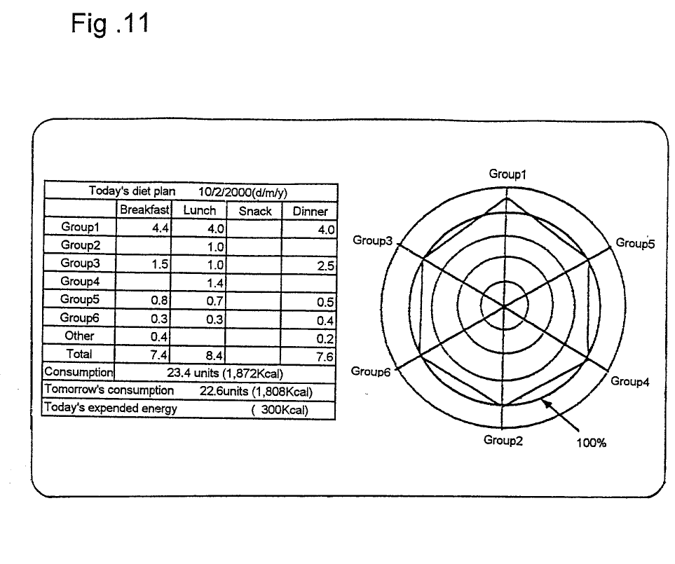

FIG. 12 is an explanatory diagram showing an example of barcodes recorded in a restaurant menu and on commercial food products;

FIG. 13 is an explanatory diagram showing an example of a portable terminal and its peripheral equipment;

FIG. 14 is a perspective view showing a portable terminal according to a second embodiment;

FIG. 15 is a block diagram showing the internal configuration of the portable terminal in FIG. 14;

FIG. 16 is a perspective view showing a portable terminal according to a third embodiment;

FIG. 17 is a block diagram showing the internal configuration of the portable terminal in FIG. 16; and

FIG. 18 is an explanatory diagram showing an example web page made accessible through the Internet by a health care facility.

### DESCRIPTION OF THE PREFERRED EMBODIMENTS

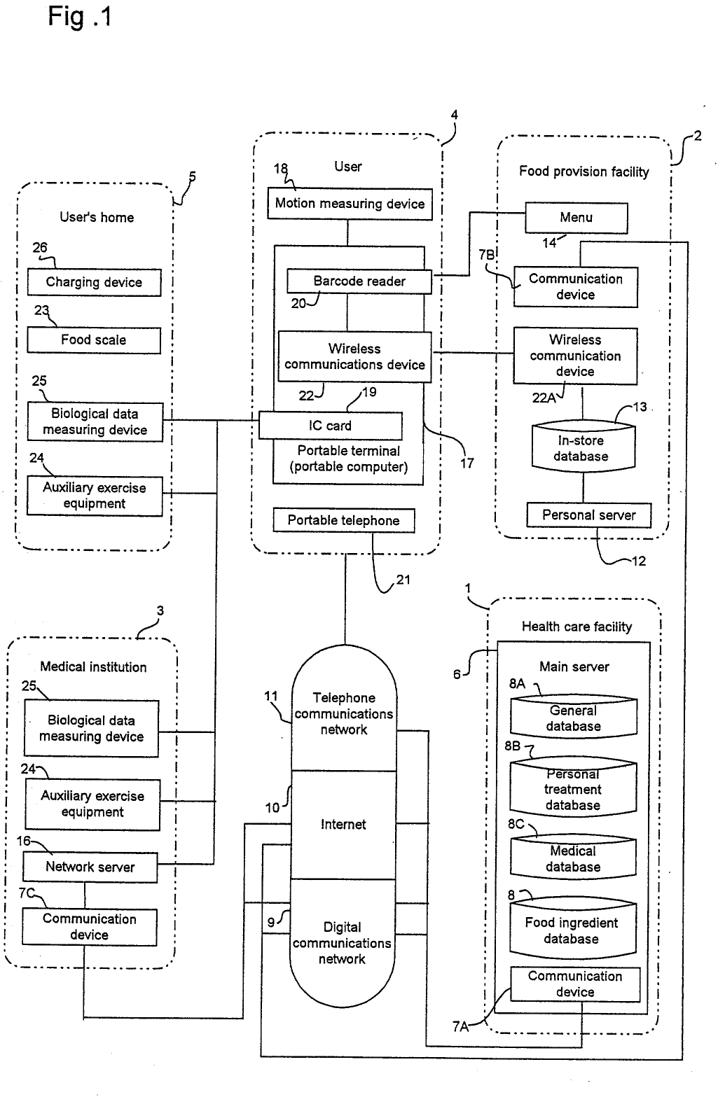

A portable self-administered health care terminal according to preferred embodiments of the present invention will be described while referring to the accompanying drawings. A portable terminal according to a first embodiment will be described first with reference to FIGS. 1□13.

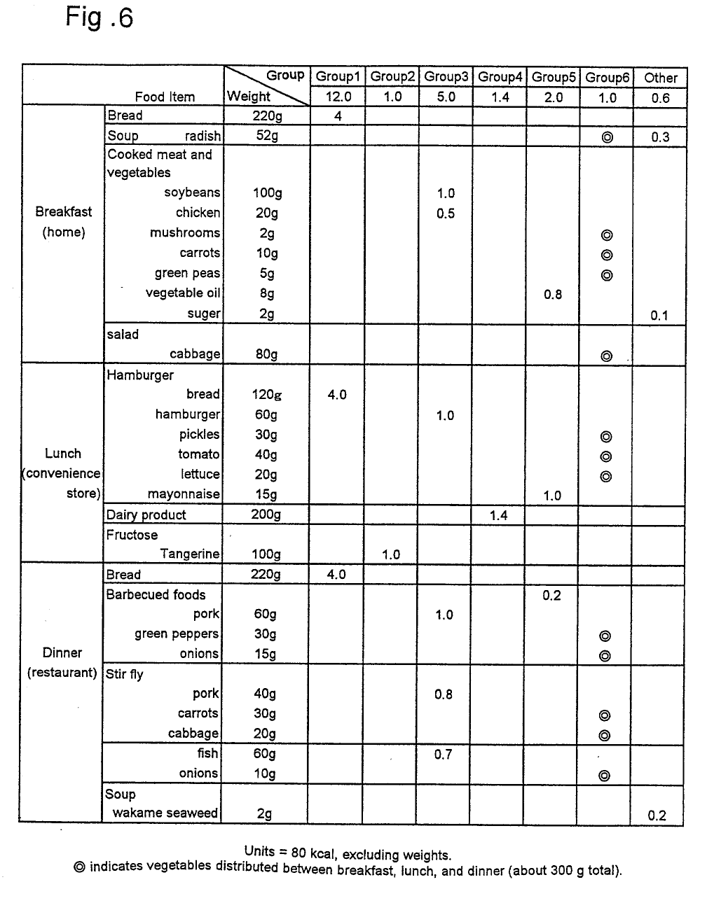

FIG. 6 is a table showing an example of energy per nutrient consumed during one day. In the diagram, Group 1 of the nutrients is foods that provide carbohydrates, such as grains; potatoes; beans, excluding soybeans and its products; and vegetables and seeds that are high in carbohydrates. Group 2 includes foods that provide carbohydrates through fructose, such as apples, tangerines, and other fruits. Group 3 includes foods that provide proteins, such as seafood; the meat of poultry, whale, and other animals and their processed goods; eggs; cheese; and soybeans and their products. Group 4 includes foods that provide calcium, such as milk and other dairy products, excluding cheese. Group 5 includes foods that provide lipids, such as oils and fats and fatty foods. Group 6 are foods that provide vitamins and minerals, such as vegetables, seaweed and mushrooms. “Other” signifies seasoning or condiments. The numbers in FIG. 6, excluding the weights, are the amounts of energy for each nutrient included in the ingredients, wherein one unit is 80 kcal.

The system of the first embodiment includes a health care facility 1 in charge of the present system; a food provision facility 2 such as a restaurant or convenience store for providing cooked foods, processed foods, and the like; a medical institution 3; a system user 4; a user's home 5 belonging to the user 4; a digital communications network 9, such as an ISDN or satellite communication circuit; an Internet 10; and a portable telephone communications network 11.

The health care facility 1 is provided with a main server 6. The main server 6 accommodates a communication device 7A, such as a router, for communicating with a communications network; a food ingredient database 8 that stores energy values for nutrients of fixed weights of various foods, such as the amounts of carbohydrates, proteins, lipids, vitamins, and minerals per 100 grams of a food; a general database 8A storing personal treatment database 8B storing treatment methods prescribed by the medical institution 3 such as daily required amounts of exercise, energy values per nutrient to consume for each breakfast, lunch and dinner, and instructions and times for taking medicines and measuring biological data, as well as records of the user's treatment status; and a medical database 8C storing procedures, prescriptions, test data from the medical institution 3.

The food provision facility 2 includes a personal server 12; an in-store database 13 of the personal server 12 that stores data regarding the energy value per nutrient found in foods provided by the food provision facility 2; a menu 14 including foods provided by the food provision facility 2; patterns or numbers, such as barcodes 15, indicating retrieval codes for looking up and extracting data from the in-store database 13 for foods that are listed in the menu 14; a communication device 7B, such as a router, for use with a communication network; and a wireless communication device 22A connected to the personal server 12.

The medical institution 3 is provided with a communication device 7C, such as a router, for communicating with a communications network; a network server 16; auxiliary exercise equipment 24, such as a treadmill; and a biological data measuring device 25, such as a sphygmomanometer, an apparatus for measuring blood sugar levels, and an apparatus for measuring body fat percentage.

The user 4 is provided with a portable terminal 17, such as a portable computer or PDA device, that can be carried by the user 4; a motion measuring device 18 that can be carried by the user 4 for measuring the amount of energy expended through exercise by detecting the user's movement; and a portable telephone 21 used for communicating with a communications network. The portable terminal 17 is provided with an IC card 19 serving as a portable data storage medium that can be read from or written to by devices provided in the auxiliary exercise equipment 24 and biological data measuring device 25; a portable barcode reader 20 that is capable of reading patterns or numbers; and a portable and detachable wireless communications device 22 for transmitting and receiving data wirelessly between the personal server 12 provided in the food provision facility 2 and the portable terminal 17.

The user's home 5 is provided with a food scale 23 for estimating the amount of nutrient energy per weight found in various foods; and a charging device 26 for charging the biological data measuring device 25 and portable terminal 17. The user's home 5 is also provided with the auxiliary exercise equipment 24 and biological data measuring device 25 found in the medical institution 3.

The self-administered health support system of the present embodiment targets people of all ages. The system is designed for patients following treatment for lifestyle-related diseases, such as diabetes, who must follow therapeutic exercises and diet regimens prescribed for treating these diseases, people who are highly susceptible to adult diseases and require management of their everyday lifestyles, and people who wish to maintain their health and follow a healthy diet plan.

As described in FIG. 1, the health care facility 1 is provided for managing the entire system. However, if many food provision facilities 2 and users 4 participate in this system, data transmissions and receptions with the health care facility 1 may greatly increase in number and become too complex. In this case, a health care facility 1 can be provided for each user 4 or for each governing region in order to decentralize the operations of the system.

In the food provision facility 2, the personal server 12 is connected to the digital communications network 9 and Internet 10 via the communication device 7B. The personal server 12 is provided with the wireless communication device 22A for transmitting and receiving data between the in-store database 13; and the wireless communications device 22 built into the portable terminal 17, which is carried by the user 4.

In the medical institution 3, the network server 16 is connected to the digital communications network 9 and Internet 10 via the communication device 7C. The network server 16 can access the databases 8B and 8C of the main server 6, which store personal treatment data such as that recorded by the user 4 and medical data respectively, and search those data bases to obtain data necessary for prescribing therapeutic exercise and a diet regimen for the relevant lifestyle-related disease. The IC card 19, which is detachably mounted in the auxiliary exercise equipment 24 or biological data measuring device 25 is used as a storage medium for inputting measurement data into the portable terminal 17.

As described above, the user 4 is provided with a barcode reader 20 capable of reading patterns or numbers. The barcode reader 20 reads data represented by the barcodes 15 on a menu of foods provided by the food provision facility 2.

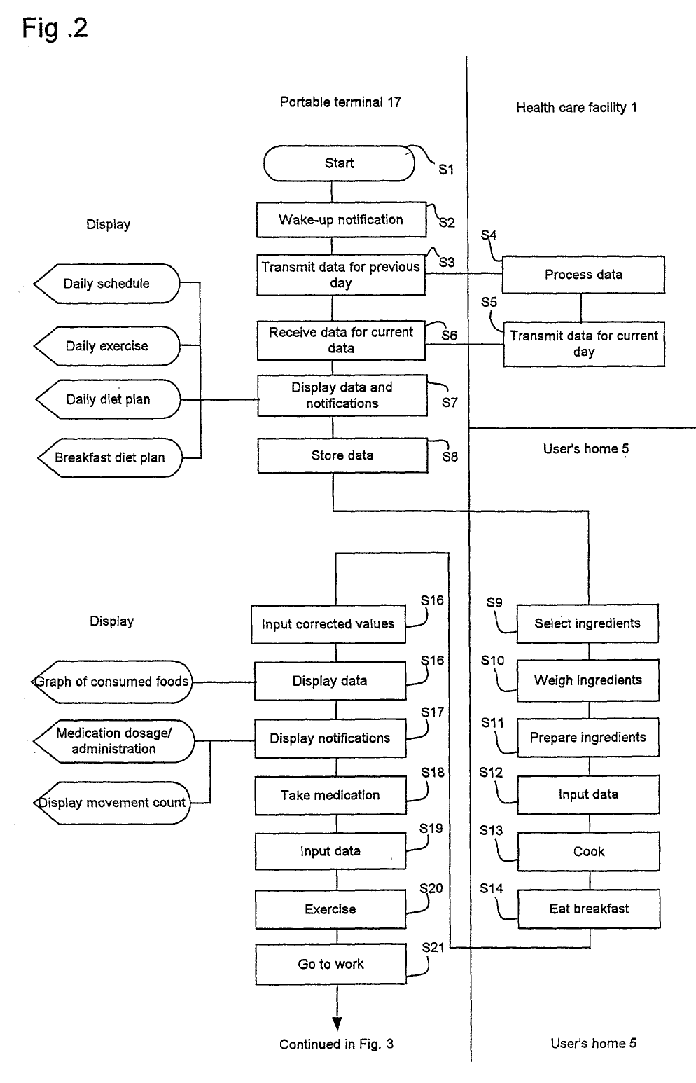

Next, an example of an operational method for a system having the above construction will be described with reference to FIGS. 2□13. The user 4 in this example is a commuting worker. First, a description of the operational method will be given for the period from early morning, when the user 4 wakes up, until the user 4 commutes to work.

(1) Before going to bed on the previous night, the user 4 connects the portable terminal 17 and motion measuring device 18 to the charging device 26. The operations of the present system begin in step S1 when charging by the charging device 26 is completed.

(2) In step S2, the portable terminal 17 notifies the user 4 that it is time to wake up by playing a melody or the like at a preset time. The portable telephone 2l automatically calls the main server 6 at the health care facility 1 and connects the portable terminal 17 thereto. Subsequently, in step S3 the portable terminal 17 transmits data collected on the previous day to the main server 6. This data includes the previous day's consumption of energy per nutrient, energy expended through exercise, and biological measurements such as blood pressure, blood sugar, and body fat percentage values.

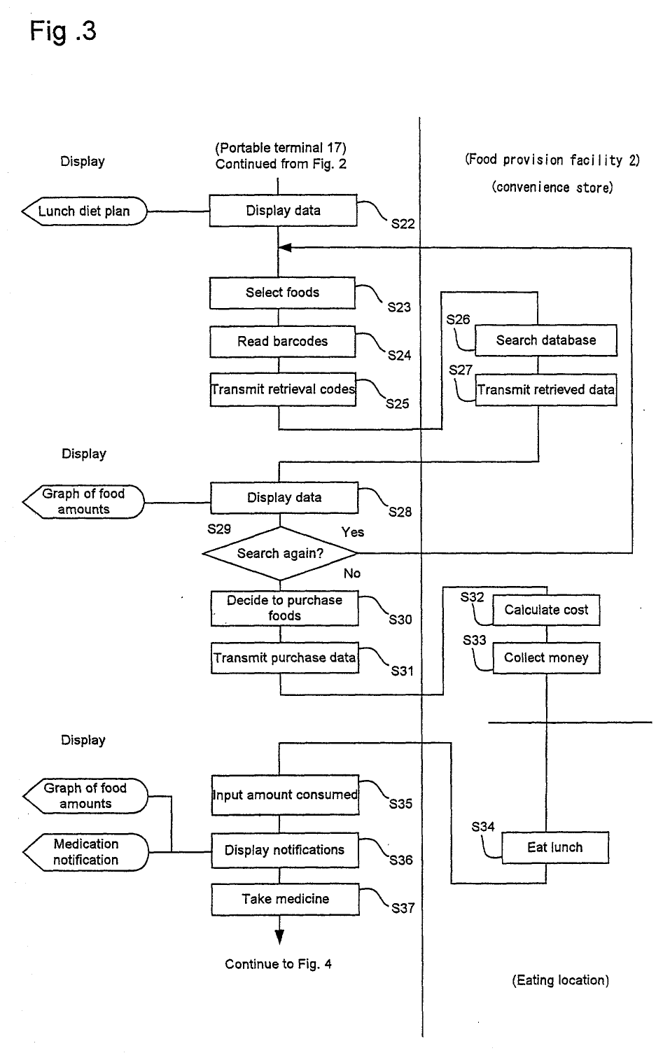

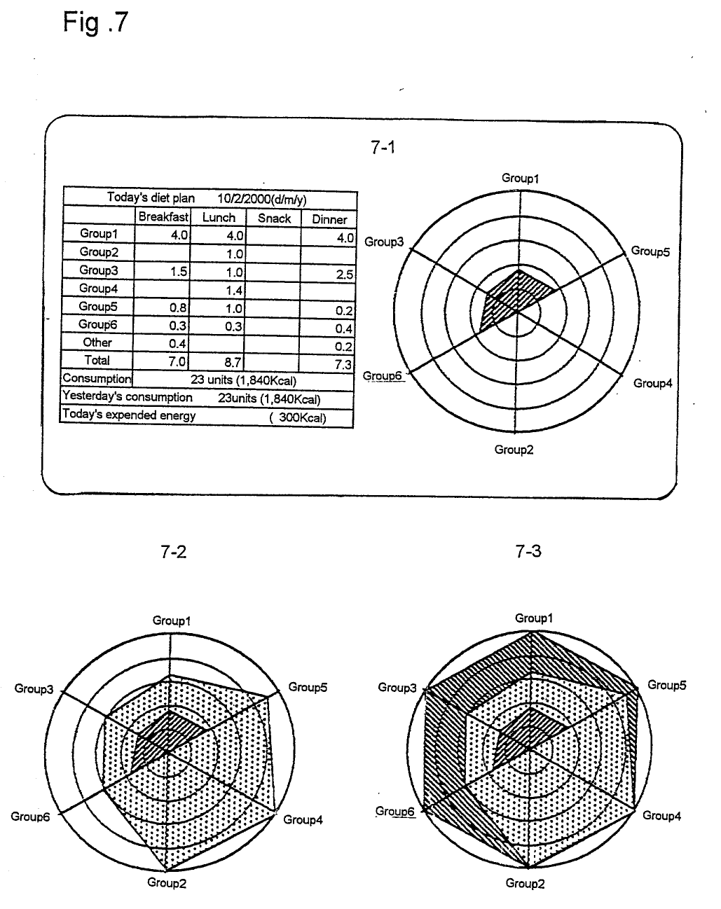

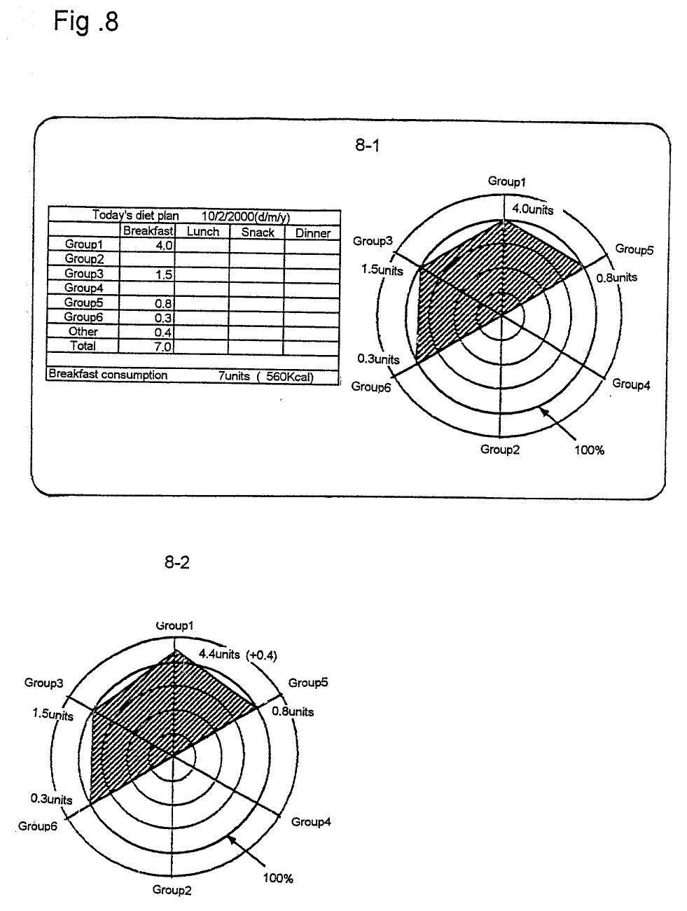

(3) In step S4, the main server 6 processes the data for the previous day received from the portable terminal 17 and calculates data for the current day. In step S5, the main server 6 transmits this data to the portable terminal 17 via the telephone communications network 11. In step S6, the portable terminal 17 receives data for the current day. In step S7, the portable terminal 17 displays the data for the current day on its display screen, including the daily schedule and the amount of exercise required for that day. The portable terminal 17 also displays a table of energy per nutrient to be consumed during one day, as shown in FIG. 7-1. In addition the portable terminal 17 displays graphs of the energy per nutrient to be consumed at breakfast (FIG. 7-1), breakfast and lunch (FIG. 7-2), and breakfast, lunch, and dinner (FIG. 7-3). The portable terminal 17 can also display energy per nutrient to be consumed at breakfast in a table and graph as shown in FIG. 8-1.

(4) These graphs and tables are displayed in the order given until finally displaying the table and graph of FIG. 8-1. In the table of FIG. 7-1, which shows daily amounts of recommended consumption, the values shown in the breakfast column (4.0, 1.5, 0.8, 0.3, 0.4, and 7.0) indicate the energy per nutrient to be consumed at breakfast. As described above, Group 1 indicates a food group that provides carbohydrates, Group 2 a food group that provides carbohydrates through fructose, Group 3 a food group that provides proteins, Group 4 a food group that provides calcium, Group 5 a food group that provides lipids, and Group 6 a food group that provides vitamins and minerals, while Other denotes spices and condiments. One unit equals 80 kcal.

(5) The displayed data is stored temporarily in the portable terminal 17 in step S8. In step S9, the user 4 or a cook selects ingredients containing nutrients listed in the table and graph of FIG. 8-1 using a food composition table that lists the energy per nutrient in the ingredients, such as the nutrient energy per 100 grams. Next, the user 4 or the cook weighs each selected ingredient with the food scale 23 in step S10 and prepares the proper amount of each ingredient in step S11 in order to gain the recommended amount of each nutrient. In step S12, the user 4 inputs the energy value per nutrient included in the prepared ingredients using input keys on the portable terminal 17. For example, the user would input 4.0 for Group 1, 1.5 for Group 3, 0.8 for Group 5, 0.3 for Group 6, 0.4 for Other, and 7.0 for the total.

(6) In step S13, the user or the cook cooks the prepared ingredients. In step S14 the user 4 consumes the prepared food for breakfast. Instep S15, the user 4 inputs correction values for the amounts of food consumed using the input keys on the portable terminal 17. For example, if the user 4 consumes 10% more bread than the recommended amount, the user 4 would input 110% for Group 1 and 100% for Groups 3, 5, and 6. After the user 4 inputs this data, the portable terminal 17 displays the graph shown in FIG. 8-2 in step S16, indicating the amount of Group 1 consumed is 4.4 units. The portable terminal 17 notifies the user 4 that the amount of Group 1 consumed is 0.4 units over the recommended amount and records the values for breakfast foods consumed.

(7) In step S17, the portable terminal 17 acknowledges that the amounts of consumed foods were input in step S15 and notifies the user 4 regarding the names and dosages of medicine to be taken, for example, 30 minutes after eating and the amounts of exercise to be performed, for example, 30 minutes of aerobic exercises. In step S18, the user 4 takes the medicine according to instructions and inputs data in step S19 into the portable terminal 17 indicating that the medicine has been taken. At this time, the names and the dosages of the medicine taken are recorded in the portable terminal 17. Wearing the portable motion measuring device 18, the user performs exercises in step S20 to burn off a prescribed amount of energy. After exercising, the user 4 commutes to the workplace while still wearing the motion measuring device 18 and begins working in step S21. The user 4 should wear the motion measuring device 18 at all times excluding those times that this is not possible, such as when bathing or swimming.

Next, operations of the portable self-administered health care terminal will be described for the period from lunch until the end of the work day, while referring to FIG. 3.

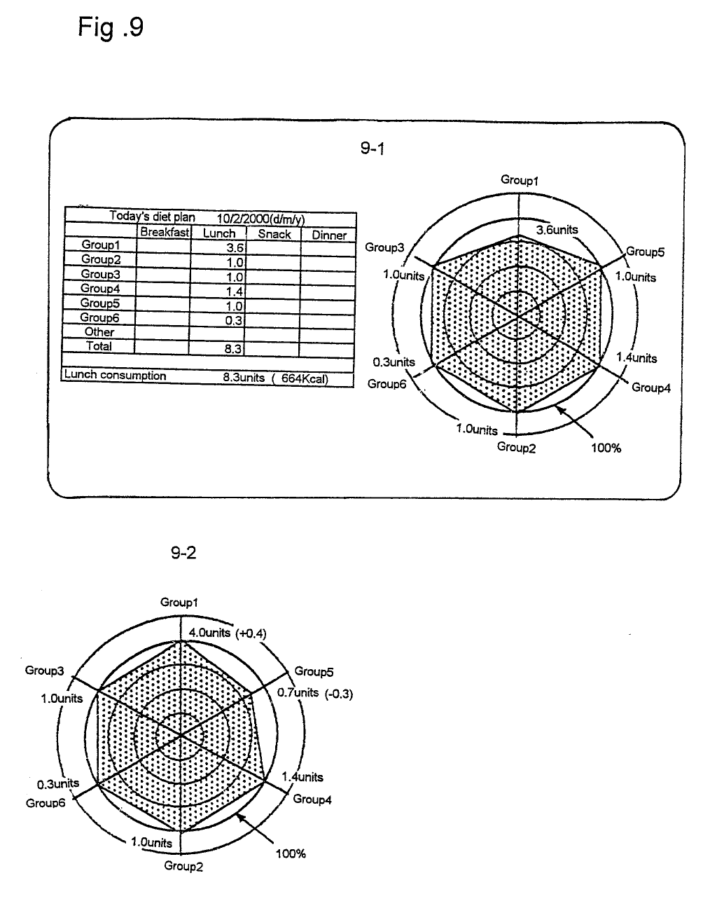

(1) In step S22, the portable terminal 17 displays a table and graph, as shown in FIG. 9-1, regarding the energy per nutrients to be consumed at lunch. Since an additional 0.4 units of bread was consumed at breakfast, the data for Group 1 to be consumed at lunch has been modified to 3.6 units.

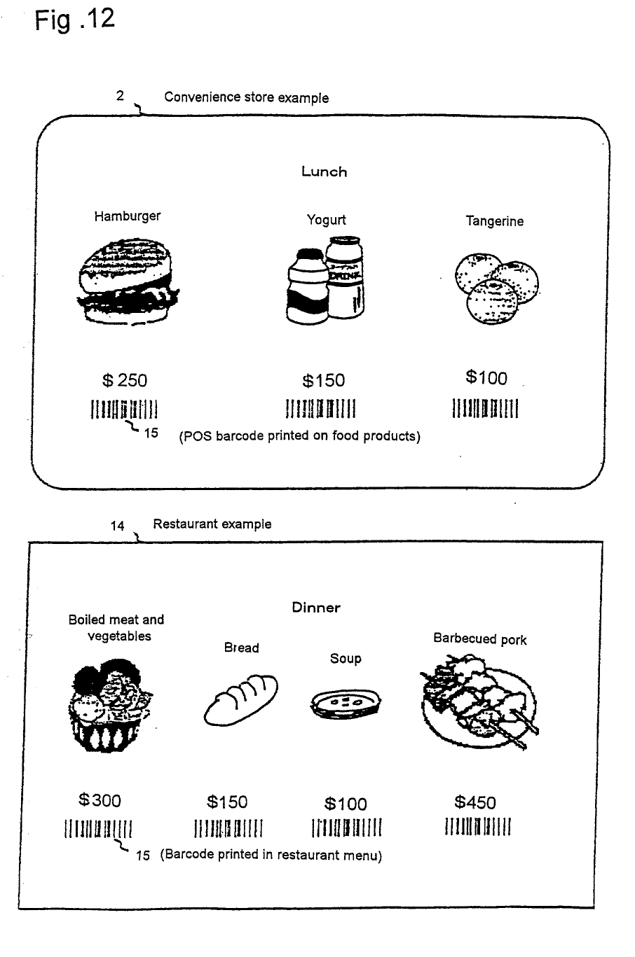

(2) In step S23, the user 4 selects a lunch capable of meeting the recommended energy per nutrient from food products displayed in a food provision facility 2, such as a convenience store or the like. In step S24, the barcode reader 20 included in the portable terminal 17 is used to read the barcodes 15 listed on the selected food products, such as a hamburger, yogurt, and tangerines shown in the example of FIG. 12.

(3) In step S25, the portable terminal 17 transmits the data read by the barcode reader 20 to the wireless communication device 22A of the personal server 12 via the wireless communications device 22. Here, the data read by the barcode reader 20 is retrieval codes for retrieving data from the in-store database 13 provided in the convenience store or the like. In step S26, the personal server 12 searches the in-store database 13 using the retrieval codes. In step s27, the personal server 12 transmits data regarding energy per nutrient in the hamburger, yogurt, and tangerines to the portable terminal 17. In step S28, the portable terminal 17 displays a graph, such as that shown in FIG. 9-2.

(4) Based on the graph shown in FIG. 9-2, the user 4 determines in step S29 whether or not to consume the selected foods. If the data in FIG. 9-2 does not meet or exceeds the amount of foods displayed in the graph of FIG. 9-1, the user 4 can select different foods by returning to step S23 or decide to purchase the foods in step S30. In this example, the user 4 will select the hamburger, yogurt, and tangerines to be consumed.

(5) In step S31, the portable terminal 17 transmits data indicating the desire to purchase the hamburger, yogurt, and tangerines to the wireless communication device 22A via the wireless communications device 22. In response, the personal server 12 calculates the cost of the selected products in step S32. In this example, the personal server 12 adds 250 yen for the hamburger, 150 yen for the yogurt, and 100 yen for the tangerines, as indicated in FIG. 12, and multiplies the result by a predetermined value, such as 2%, for a result of 10 yen. This calculated data is transmitted to the register in the convenience store as a fee for providing data and is added to the total purchase. The money is collected from the user 4 in step S33.

(6) In step S34, the user 4 eats lunch, while recognizing that 10% less of the hamburger bun should be consumed because the energy per nutrient contained in the lunch exceeds the recommended value for Group 1 by 0.4 units. After eating lunch, the user inputs in step 35 the amount of food consumed using the input keys on the portable terminal 17. For example, if the user 4 eats the entire hamburger bun belonging to the food group 1 without following the recommendation to reduce the hamburger bun by 10% but does not eat mayonnaise included in Group 5, then the user inputs 100% for Groups 1, 2, 3, and 6 and 70% for Group 4.

(7) The portable terminal 17 displays a graph of food amounts consumed at lunch, showing +0.4 units for Group 1 and −0.3 units for Group 5, and saves the data. Next, the portable terminal 17 notifies the user in step S36 of the names and dosages of medicines to be taken 30 minutes after lunch, for example. In step S37, the user takes the recommended medicine and then inputs into the portable terminal 17 data indicating that the medicine has been taken. The names and amounts of medicine taken are recorded in the portable terminal 17.

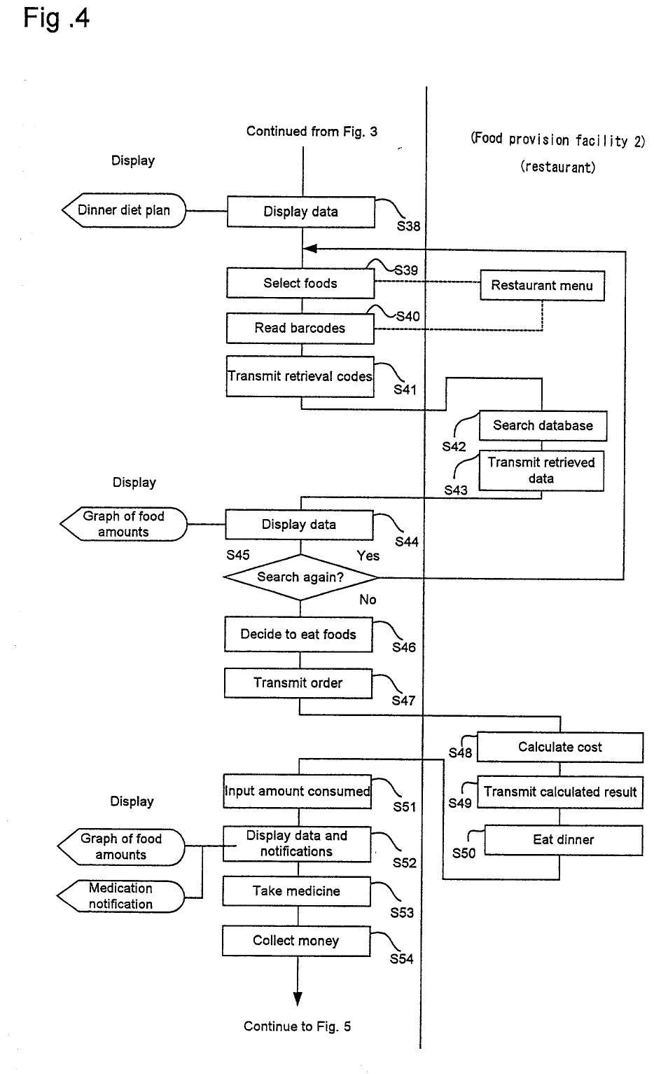

Next, operations for the portable self-administered health care terminal will be described for the period from the end of work until the user returns home, with reference to FIG. 4.

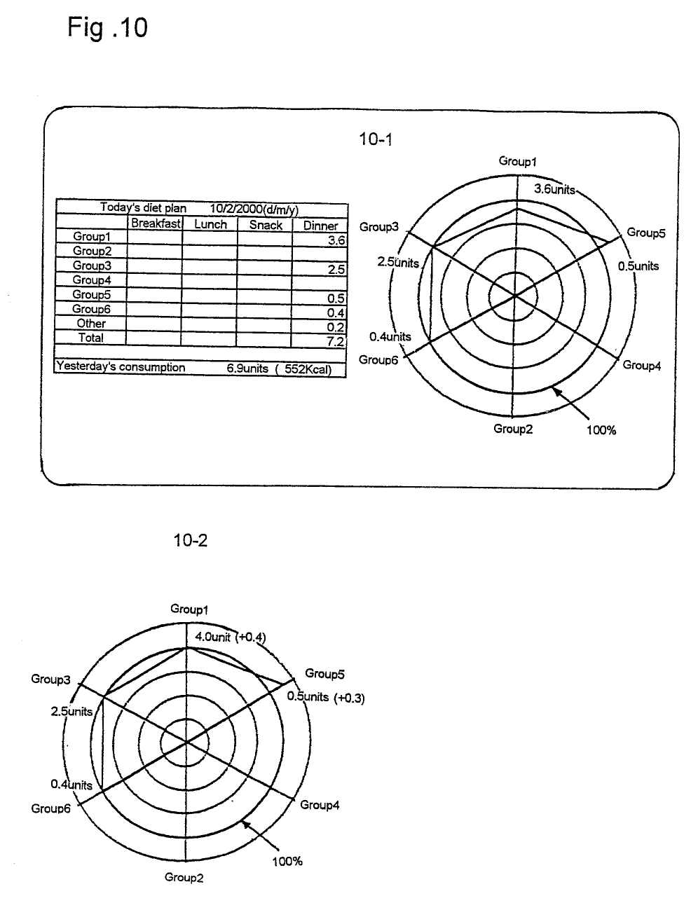

(1) In step S38, the portable terminal 17 notifies the user 4 of energy per nutrient that should be consumed for dinner by displaying a table and graph, as shown in FIG. 10-1. Since an excess of 0.4 units in Group 1 and a deficit of 0.3 units in Group 5 were consumed during lunch, the data in FIG. 10-1 has been modified accordingly. That is, the 4.0 units of Group 1 that is normally consumed has been decreased to 3.6 units, while the normal consumption of 0.2 units in Group 5 has been increased to 0.5 units. In this example, the user 4 eats dinner according to the amounts of nutrients listed at a restaurant serving as the food provision facility 2.

(2) In step S39, the user 4 selects foods from the products listed in the menu 14 that is provided at the restaurant. In this example, the user 4 selects boiled meat and vegetables, bread, soup, and barbecued pork, as shown in FIG. 12. In step S40, the barcode reader 20 connected to the portable terminal 17 is used to read the barcode 15 for each of the food items selected from the menu 14.

(3) In step S41, the portable terminal 17 transmits the data read by the barcode reader 20 to the wireless communication device 22A of the personal server 12 via the wireless communications device 22. Here, the data read by the barcode reader 20 is retrieval codes for retrieving data from the in-store database 13 provided in the restaurant. In step S42, the personal server 12 searches the in-store database 13 using the retrieval codes. In step s43, the personal server 12 transmits data regarding energy per nutrient in the boiled meat and vegetables, bread, soup, and barbecued pork to the portable terminal 17. In step S44, the portable terminal 17 displays a graph, such as that shown in FIG. 10-2.

(4) Based on the graph shown in FIG. 10-2, the user 4 determines in step S45 whether or not to consume the selected foods. If the data in FIG. 10-2 does not meet or exceeds the amount of foods displayed in the graph of FIG. 10-1, the user 4 can select different foods by returning to step S39 or decide to purchase the foods in step S46. In this example, the user 4 will select the boiled meat and vegetables, bread, soup, and barbecued pork.

(5) In step S47, the portable terminal 17 transmits data indicating the desire to purchase the boiled meat and vegetables, bread, soup, and barbecued pork to the wireless communication device 22A via the wireless communications device 22. In response, the personal server 12 calculates the cost of the selected products in step S48. In this example, the personal server 12 adds 300 yen for the boiled meat and vegetables, 150 yen for the bread, 100 yen for the soup, and 450 yen for the barbecued pork, as indicated in FIG. 12, and multiplies the result by a predetermined value, such as 2%, for a result of 20 yen. This calculated data is transmitted to the register in the food provision facility 2 in step S49 as a fee for providing data.

(6) In step S50, the user 4 eats dinner, while recognizing that 10% less of the bread should be consumed because the energy per nutrient contained in the dinner exceeds the recommended value for Group 1 by 0.4 units. After eating dinner, the user inputs in step S51 the amount of food consumed using the input keys on the portable terminal 17. For example, if the user 4 eats all the bread, which belongs to the Group 1, without following the recommendation to reduce the bread consumption by 10%, then the user inputs 100% for Groups 3, 5, and 6 and 110% for Group 1.

(7) The portable terminal 17 displays a graph of food amounts consumed at dinner, showing +0.4 units for Group 1 and +0.3 units for Group 5, and saves the data. Next, the portable terminal 17 notifies the user in step S52 of the names and dosages of medicines to be taken 30 minutes after dinner, for example. In step S53, the user takes the recommended medicine and then inputs into the portable terminal 17 data indicating that the medicine has been taken. The names and amounts of medicine taken are recorded in the portable terminal 17. When settling the bill, the food provision facility 2 adds a 20 yen data fee to the total bill of 1,000 yen and collects the money from the user 4 in step S54.

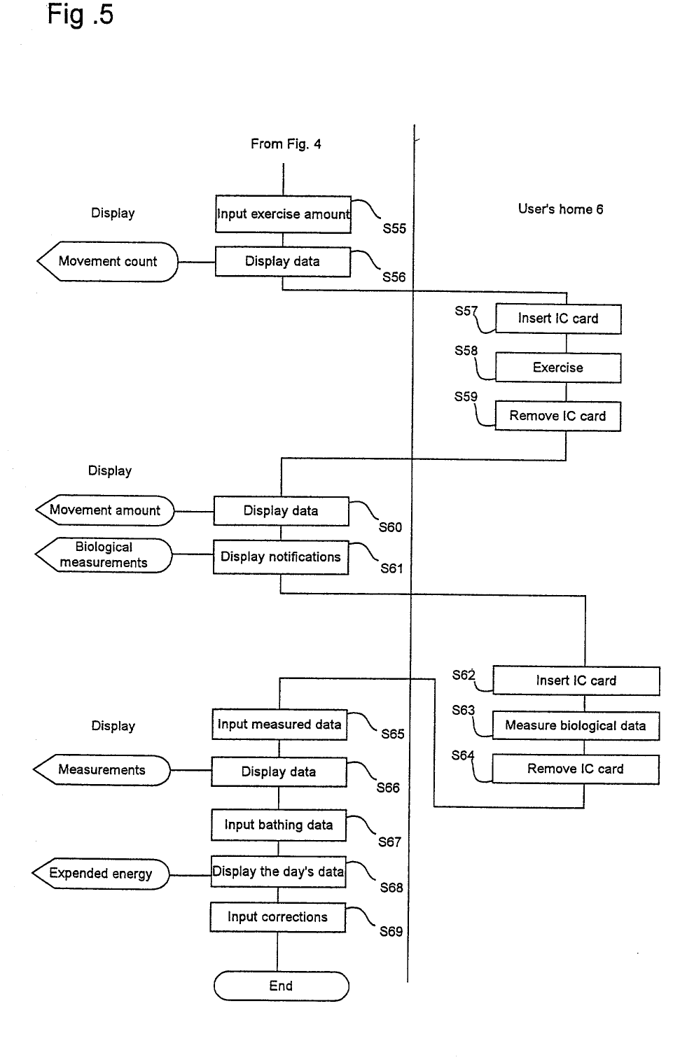

Next, operations of the portable self-administered health care terminal will be described for the period after the user returns home until the user retires to bed, while referring mainly to FIG. 5.

(1) After returning home, the user 4 confirms the amount of energy expended through exercise during daily activities from morning until the present time, based on data displayed in the motion measuring device 18. The user 4 has been constantly wearing the motion measuring device 18 since waking up. In step S55, the user 4 inputs this data into the portable terminal 17. The expended energy is recorded in the portable terminal 17. In step S56, the portable terminal 17 displays a comparison of the amount of energy expended through energy that was prescribed by the medical institution 3 or the like and the amount of expended energy input by the user, indicating how much the user has gone over or under the daily prescribed amount of expended energy.

(2) For example, if the user is 50 kcal short of the prescribed amount of expended energy based on this display, the user 4 inserts the IC card 19 into the auxiliary exercise equipment 24 in step S57. The auxiliary exercise equipment 24 is a treadmill or the like provided in the user's home 5. In step S58, the user 4 exercises for a prescribed time, such as 30 minutes. After exercising, the user removes the IC card 19 from the treadmill in step S59 and inserts the IC card 19 into the portable terminal 17. By doing so, the amount of exercise recorded in the IC card 19 is input into the portable terminal 17 and displayed on the display screen in step S60.

(3) Next, the portable terminal 17 displays notifications in step S61 prompting the user to perform biological measurements, such as measurements of weight and body fat percentage. In step S62, the user 4 inserts the IC card 19 into the biological data measuring device 25, which is a weight/body fat scale or the like and measures his or her weight and body fat percentage in step S63. In step S64, the user removes the IC card 19 from the biological data measuring device 25 after completing the measurements and inserts the IC card 19 into the portable terminal 17. As a result, the biological data is input into the portable terminal 17 in step S65 and displayed on the screen in step S66.

(4) Before going to bed, the user 4 inputs any data that has not yet been input and energy expended during times in which the motion measuring device 18 could not be worn, such as during bathing time, strenuous movement, or swimming in step S67. For example, the user might enter 30 minutes for bathing time using the input keys on the portable terminal 17. In step S68, all data collected during the current day is displayed on the screen of the portable terminal 17 for confirmation. If there is any incorrect data, revised data is input in step S69. Finally, the portable telephone 21 connected to the portable terminal 17 and the motion measuring device 18 are connected to the charging device 26, thereby completing the operations of the day.

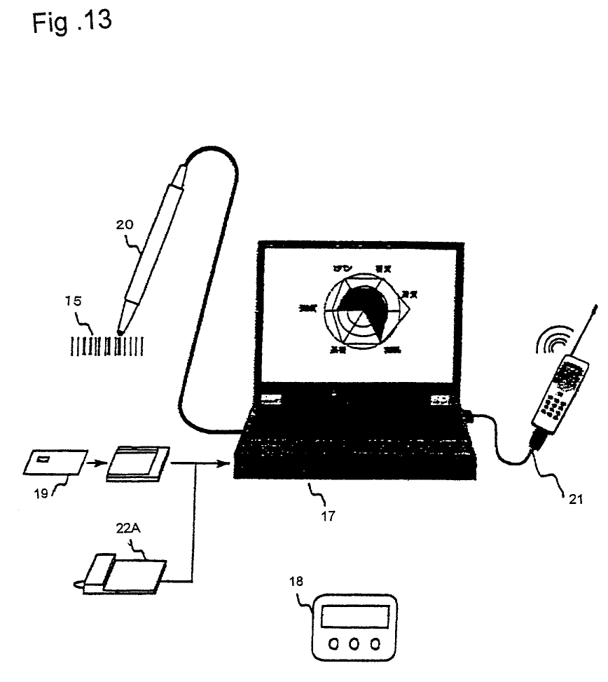

FIG. 13 shows the portable terminal carried by the user and its peripheral devices. As shown in the diagram, the portable terminal 17 is a compact personal computer, PDA, or the like. The IC card 19 is mounted in an IC card reader/writer 19A and the IC card reader/writer is detachably mounted in the portable terminal 17. The pen-type barcode reader 20 is provided for reading the barcodes 15 provided in menus of food provision facilities. Other auxiliary devices include the portable telephone 21, the portable motion measuring device 18, and the detachable wireless communications device 22.

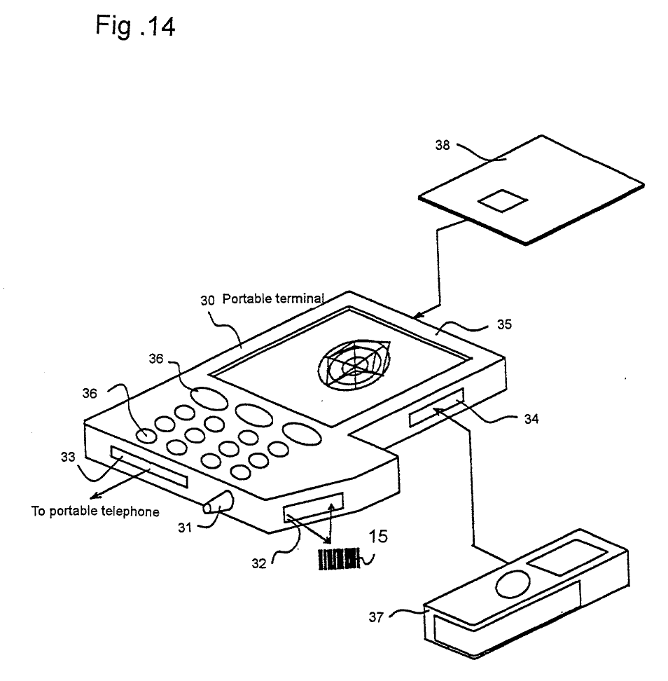

FIG. 14 shows a portable terminal according to a second embodiment of the present invention. In this example, a barcode reader, a motion measuring device, a wireless communications device, and an IC card reader/writer are integrally built into the portable terminal. As shown in FIG. 14, a portable terminal 30 is provided with an antenna 31 for the wireless communications device used to transmit and receive data at a food provision facility; a reading unit 32 of the barcode reader; a connection terminal 33 for the portable telephone; a connection terminal 34 for connecting the motion measuring device to the portable terminal 30; a liquid crystal display 35; operation keys 36; a detachable motion measuring device 37; and an IC card 38 serving as a data storage medium. Integrally combining all the devices in this way increases the portability of the terminal.

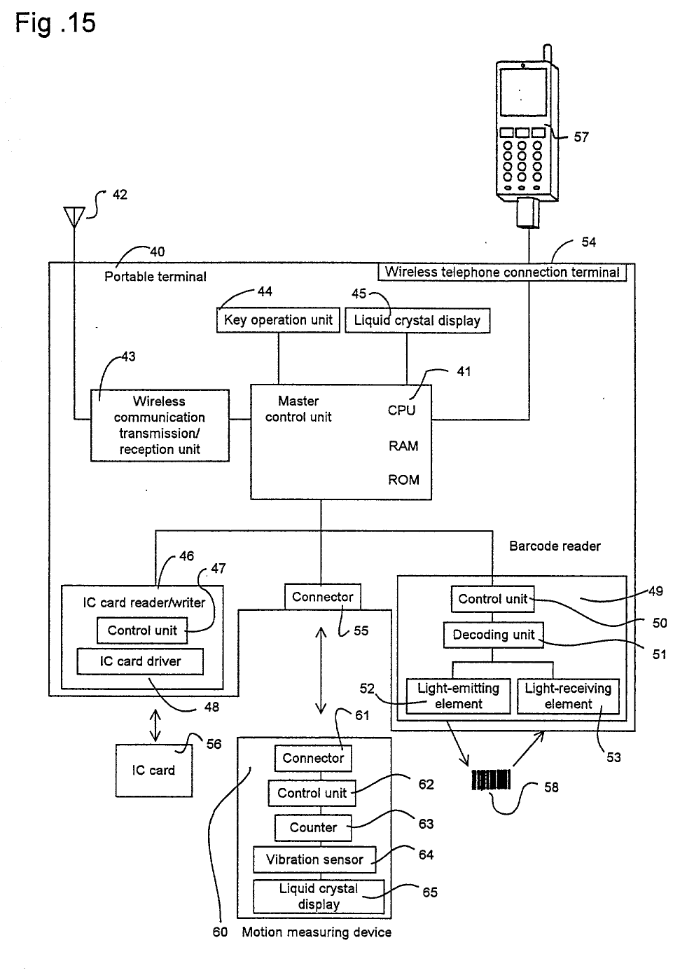

FIG. 15 is a block diagram showing the internal configuration of a portable terminal 40, such as that shown in FIG. 14. As shown in the diagram, the 40 is provided with a master control unit 41 for controlling the overall operations of the portable terminal 40; an antenna 42; a wireless communication transmission/reception unit 43 for transmitting and receiving data between a food provision facility; a key operation unit 44 for operating the portable terminal 40; a liquid crystal display 45; an IC card reader/writer 46; a control unit 47 for the IC card reader/writer 46; an IC card driver 48 for reading data from and writing data to an IC card; a barcode reader 49; a control unit 50 for the barcode reader 49; a decoding unit 51; a light-emitting element 52; a light-receiving element 53; a connection terminal 54 for the portable terminal 40; a connector 55 for connecting the detachable motion measuring device; an IC card 56 serving as a data storage medium; a portable telephone 57 connected to the portable terminal 40; barcodes 58 included in a menu provided at food provision facilities; a detachable motion measuring device 60; a connector 61; a control unit 62 for controlling the motion measuring device 60; a vibration sensor 64 for detecting motion; a counter 63 for counting signals from the vibration sensor 64; and a liquid crystal display 65 for displaying the amount of motion.

In the portable terminal 40 of FIG. 15, the master control unit 41 accommodates a CPU, a ROM, and a RAM for controlling the overall operations of the portable terminal 40. The wireless communication transmission/reception unit 43 is a circuit for performing wireless communications with a server at a food provision facility. The wireless communication transmission/reception unit 43 transmits signals requesting data for energy per nutrient in foods provided at the facility to the server of the same and receives data from that server in response.

The key operation unit 44 is used for selecting operation modes of the portable terminal 40 and for inputting data. The liquid crystal display 45 displays amounts of energy including in consumed foods, amounts of energy expended during exercise, and various notifications, such as instructions for taking medicine.

When a IC card 56 is mounted in the IC card reader/writer 46, the IC card driver 48 provided in the IC card reader/writer 46 writes data to the IC card 56 and reads data therefrom. The IC card 56 serves as a data storage medium for inputting data regarding amounts of movement through auxiliary exercise equipment such as a treadmill and data regarding blood pressure and blood sugar levels based on biological data measuring devices into the portable terminal 40.

The barcode reader 49 serves to read the barcodes 58 provided in menus at food provision facilities. The light-emitting element 52 of the barcode reader 49 comprises an LED array. The light-receiving element 53 comprises CCDs. Light emitted from the light-emitting element 52 reflects off the barcodes 58 and enters the light-receiving element 53. The decoding unit 51 analyzes the pattern of reflected light detected by the light-receiving element 53 to extract data included in the barcodes and transmits this data to the master control unit 41.

The motion measuring device 60 is detachably mounted on the portable terminal 40 via the connector 55 and connector 61. The vibration sensor 64 built into the motion measuring device 60 detect spacing and other human movement. The counter 63 counts the number of paces. This count is converted to expended energy through exercise and transmitted to the master control unit 41. Subsequently, the count is displayed on the liquid crystal display 65.

This portable terminal 40 can be connected to a communications network in order to perform communications with a server or the like at a health care facility. To do this, the portable telephone 57 is connected to the connection terminal 54 to transmit and receive data.

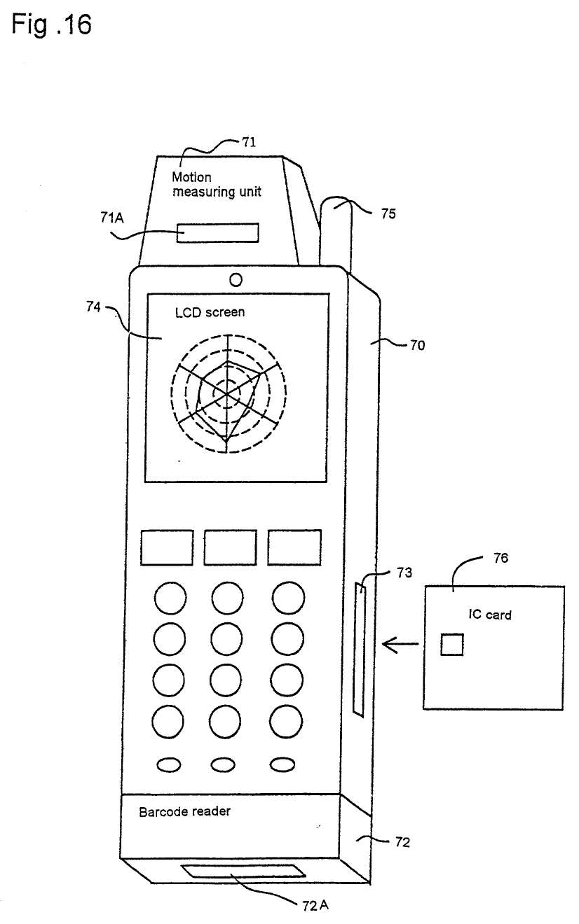

FIG. 16 shows the construction of a portable terminal according to a third embodiment of the present invention. In this embodiment, the portable terminal and portable telephone are combined as one integral unit. Further, this unit is equipped with a motion measuring device, barcode reader, wireless communication device for communicating with food provision facilities, and IC card reader/writer. As shown in FIG. 16, a portable terminal 70 includes a motion measuring unit 71 employing a vibration sensor; a motion display unit 71A disposed in the motion measuring unit 71; a barcode reader 72; a barcode reading unit 72A disposed in the barcode reader 72; an IC card slot 73; an LCD screen 74; an antenna 75; and an IC card 76, serving as a data storage medium. In this embodiment, all functions necessary for the portable terminal are integrated in the terminal itself. Combining all components into a compact package enhances the convenience of the portable terminal.

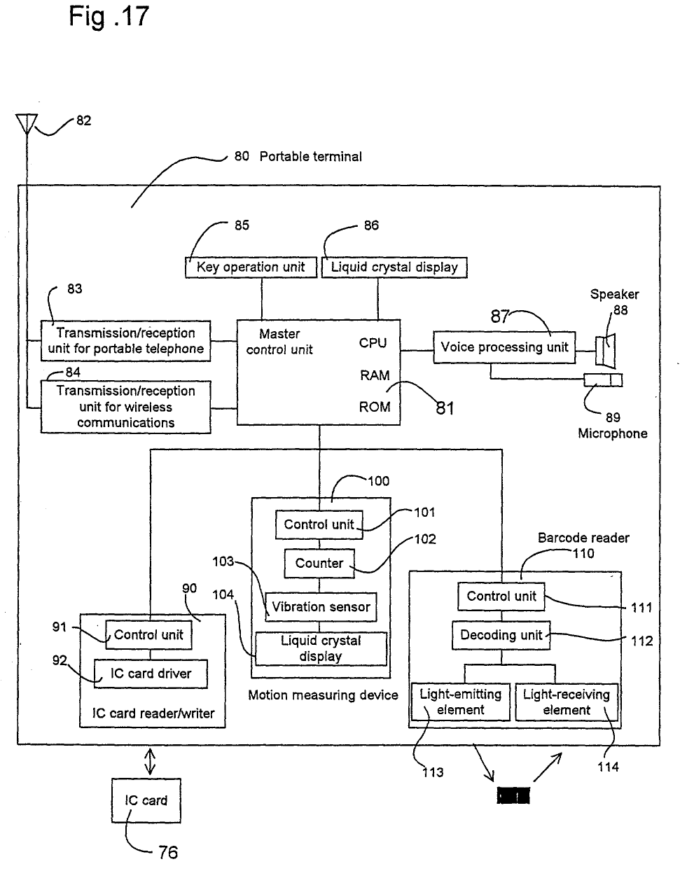

FIG. 17 is a block diagram showing the internal configuration of a portable terminal, such as that shown in FIG. 16. As shown in the diagram, a portable terminal 80 is provided with a master control unit 81 for controlling the overall operations of the portable terminal 80; an antenna 42; a transmission/reception unit 83 for the portable telephone; a wireless communications transmission/reception unit 84 for transmitting and receiving data between food provision facilities; a key operation unit 85; a liquid crystal display unit 86; a voice processing unit 87 for the portable telephone; a speaker 88; a microphone 89; an IC card reader/writer 90; a control unit 91 for controlling the IC card reader/writer 90; an IC card driver 92; a motion measuring unit 100; a control unit 101 for controlling the motion measuring unit 100; a vibration sensor 103 for detecting motion; a counter 102 for counting signals from the vibration sensor 103; a liquid crystal display unit 104 for displaying the amount of motion; a barcode reader 110; a control unit 111 for the barcode reader 110; a decoding unit 112; a light-emitting element 113; and a light-receiving element 114.

Compared to the configuration shown in FIG. 15, the portable terminal 80 shown in FIG. 17 has added the built-in function of the portable telephone. In addition, the motion measuring unit 100 is no longer detachable, but is integrally provided in the portable terminal 80. The added functional units for the portable telephone unit are well known in the art. All other components are the same as those described in FIG. 15. Therefore, a description of these parts will be omitted here.

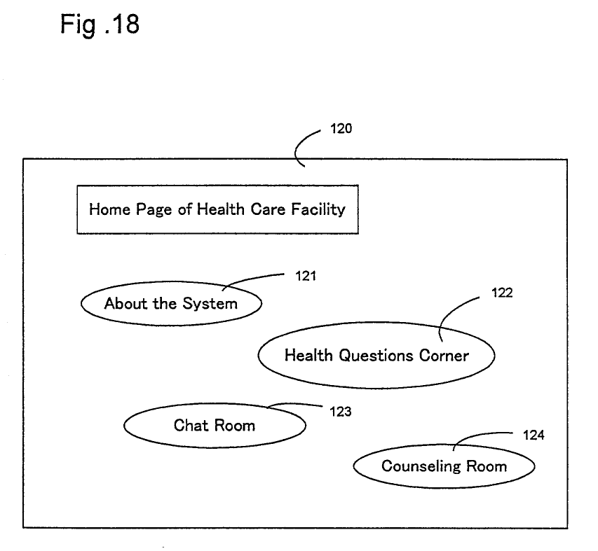

FIG. 18 shows an example of a web page 120 made available by the health care facility on the Internet. This web page 120 is provided for assisting users of the present system by providing useful data, guidance for the user 4; and counseling to help the user deal with any emotional problems. The user 4 can display the web page 120 using the portable terminal 17 or an Internet-capable portable telephone 21.

As shown in FIG. 18, the web page 120 of the health care facility includes various links to information, such as About the System 121, Health Questions Corner 122, Chat Room 123, and Counseling Room 124. By clicking on the link About the System 121, the relevant web page (not shown) will open, displaying information and instructions for the health care facility 1 and data for users of the system. By clicking on the link Health Questions Corner 122, the relevant web page (not shown) will open, enabling users to ask questions regarding health that will be answered by the health care facility. By clicking on the link Chat Room 123, the relevant web page (not shown) will open, enabling users to exchange opinions with and encourage other patients having a similar disease. By clicking on the link Counseling Room 124, the relevant web page (not shown) will open, wherein an expert will provide counseling for users having problems continuing treatment, in order to alleviate their emotional burdens and enhance the effects of the treatment.

The health care facility can also provide via e-mail data regarding the user's self-management status to a health insurance institute with which the user is contracted, upon receiving the user's permission, to enable the user to receive a special insurance contract. The user may be able to receive a discount on insurance fees with the condition that the user agrees to continue following the self-managed health plan. Since a person can substantially reduce medical bills by the prevention or curing of diseases through self-care, insurance companies are able to reduce insurance fees for such fees. Insurance companies can also require the practice of self-care as a condition for entering a contract with users who already have a disease. Hence, the health care facility can provide data on the self-care status of a user having an insurance contract to show the fulfillment of these conditions.

While the invention has been described in detail with reference to specific embodiments thereof, it would be apparent to those skilled in the art that many modifications and variations may be made therein without departing from the spirit of the invention, the scope of which is defined by the attached claims.

With the portable self-administered health care terminal and a self-administered health support system of the present invention, a health care facility can perform focused and specialized management of a user's exercise and dietary treatment. Hence, by caring a portable terminal, each user can receive detailed treatment directions. Further, since it is possible to obtain accurate treatment records through the portable terminal, a medical institute can offer more accurate prescriptions, while the users can concentrate on their treatment without concerns for reliability.

With the present invention, medical institutes can gather accurate data regarding biological measurements for the user, amounts of energy expended during exercise, and amounts of energy per nutrient in foods consumed that is indispensable for prescribing therapeutic exercise and diet regimens used for treating such lifestyle-related diseases as diabetes. Hence, the present invention increases the effects of treating lifestyle diseases that are currently on the rise and can suppress the outbreak and spreading of such diseases. These effects also lead to reduced medical costs.

The present invention can automatically and accurately record amounts of energy per nutrient in foods consumed at food provision facilities. When eating at restaurants, users have conventionally had to estimate the types and weights of ingredients in foods on the restaurant menu, estimate the amounts of energy per nutrient included in these foods based on a food composition table, and record this calculated data by hand. The present invention frees the user from this work.

The present invention also prompts the user through the portable terminal to take medicines and measure biological data. Further, the dosage and administration of the medicine and the biological data measurements are recorded in the portable terminal. Accordingly, the administrations of medicines and measurements can be reliably performed and accurately recorded.

The present invention uses a portable motion measuring device to measure the amount of energy expended through exercise by detecting the motion of the user. As a result, the amount of energy expended through exercise can be accurately recorded.

Through the use of a portable storage device such as an IC card, the present invention facilitates the inputting of data from biological data measuring devices for measuring blood pressure and blood sugar levels and amounts of energy expended on auxiliary exercise equipment such as treadmills into the portable terminal, thereby decreasing the load on the user.

According to another aspect of the present invention, the convenience of the portable terminal can be enhanced by integrating the wireless communication device for communicating with the food provision facility, the motion measuring device, the barcode reader, and the IC card reading/writing device into the portable terminal itself.

According to another aspect of the present invention, the convenience of the portable terminal can be even further enhanced by integrating the portable terminal with a portable terminal, in addition to integrating the wireless communication device for communicating with the food provision facility, the motion measuring device, the barcode reader, and the IC card reading/writing device into the portable terminal itself.

With the self-administered health support system of the present invention, web pages for the health care facility can be provided for responding to user's questions, providing counseling in response to user's emotional concerns during treatment, and providing the means for bulletin boards and chat rooms for user's to exchange opinions with other patients. Accordingly, the present invention increases the percentage of patients who fulfill their goals during long periods of treatment without suffering setbacks.

Since, the self-administered health support system is also provided with a system for providing data for special insurance contracts, the user may be able to receive a discount on insurance fees with the condition that the user agrees to continue following the self-managed health plan. Since a person can substantially reduce medical bills by the prevention or curing of diseases through self-care, insurance companies are able to reduce insurance fees for such fees. Insurance companies can also require the practice of self-care as a condition for entering a contract with users who already have a disease.

What is claimed is:

1. A portable self-administered health care terminal for supporting self-management of diet and exercise, comprising:
communication means for exchanging data with external equipment via a communications network;
display means for receiving and displaying data regarding recommended amounts of energy per nutrient to be consumed, daily required exercise, and other data required for treatment from a server at a health care facility for supporting treatment for users with portable terminals;
input means for inputting data concerning the energy contained in consumed foods and energy expended through exercise;
reception means for receiving data regarding amounts of nutrient energy contained in foods about to be consumed from a server at a food provision facility equipped with a database that stores amounts of nutrient energy contained in foods that the facility provides; and
transmission means for transmitting data regarding energy contained in consumed foods and energy expended by exercise to the server at the health care facility.

2. A portable self-administered health care terminal for supporting self-management of diet and exercise, comprising:
communication means for exchanging data with external equipment via a communications network;
display means for receiving and displaying data regarding amounts of daily required exercise, nutrient energy that should be consumed, instructions on dosage and administration of medicine, instructions on taking biological measurements, and other data required for treatment from a server at a health care facility for supporting treatment for users with portable terminals;
input means for inputting with key operations data regarding the nutrient energy in consumed foods;
input means for inputting with key operations data regarding the energy expended in exercise;
transmission means for reading patterns or numbers from a restaurant menu and transmitting that data to a server of a food provision facility in order to receive data regarding amounts of nutrient energy contained in foods about to be consumed from a server at a food provision facility equipped with a database that stores amounts of nutrient energy contained in foods that the facility provides;
reception means for receiving data regarding amounts of nutrient energy contained in foods about to be consumed from the server at the food provision facility;
input means for inputting data from a motion measuring device for detecting movement of a human body during exercise and measuring the amount of energy expended;
input means for inputting data from a sphygmomanometer and other biological data measuring devices;
prompting means for prompting the patient to take medicine and perform biological measurements;
display means for displaying data for the amount of energy consumed through food and data for the amount of energy expended through exercise; and
transmission means for transmitting data regarding energy consumed through foods, energy expended through exercise, instructions on dosage and administration of medicine and instructions on taking biological measurements to the server at the health care facility.

3. A portable self-administered health care terminal for supporting self-management of diet and exercise, comprising:
communication means for exchanging data with external equipment via a communications network, wireless telephone network, and wireless telephone;
display means that uses the functions of a wireless telephone to receive and display data regarding amounts of daily required exercise, nutrient energy that should be consumed, instructions on dosage and administration of medicine, instructions on taking biological measurements, and other data required for treatment from a server at a health care facility for supporting treatment for users with portable terminals;
input means for inputting with key operations data regarding the nutrient energy in consumed foods;
input means for inputting with key operations data regarding the energy expended in exercise;
transmission means for reading patterns or numbers from a restaurant menu and transmitting that data via a wireless communication device to a server of a food provision facility in order to receive data regarding amounts of nutrient energy contained in foods about to be consumed from a server at a food provision facility equipped with a database that stores amounts of nutrient energy contained in foods that the facility provides;
reception means for receiving data via a wireless communication device from the server at the food provision facility regarding amounts of nutrient energy contained in foods about to be consumed;
means for displaying food consumption after determining the correctness thereof based on data regarding amounts of nutrient energy contained in foods about to be consumed and the recommended daily amounts of nutrient energy received from the health care facility;
input means for inputting data from a motion measuring device for detecting movement of a human body during exercise and measuring the amount of energy expended;
means for recording data from a sphygmomanometer and other biological data measuring devices and data from a treadmill and other auxiliary exercise equipment onto an IC card or other portable storage medium, and for inputting data stored on the portable storage medium into the portable terminal via a storage medium reading/writing device provided in the portable terminal;
prompting means for prompting the patient to take medicine and perform biological measurements;
display means for displaying data for the amount of energy consumed through food and data for the amount of energy expended through exercise; and
transmission means for using the functions of a wireless telephone to transmit data regarding energy consumed through foods, energy expended through exercise, instructions on dosage and administration of medicine and instructions on taking biological measurements to the server at the health care facility.

4. A portable self-administered health care terminal as recited in claim 2 or 3, wherein the patterns provided in the menus at the food provision facility are barcodes and a barcode reader is used to input barcode data into the portable terminal.

5. A portable self-administered health care terminal as recited in claim 3 or 4, wherein the portable terminal is provided with one or all of a wireless telephone, a wireless communication device for transmitting and receiving data between the food provision facility, a motion measuring device, a device for reading the patterns or numbers recorded in the menus, and a device for reading from and writing to the portable recording medium.

6. A portable self-administered health care terminal as recited in any of claims 1 through 5, wherein the communications network is the Internet.

7. A self-administered health support system for supporting a patient's self-administration of diet and exercise, comprising:
a server at a health care facility;
a server at a food provision facility;
a server at a medical institute;
a portable terminal carried by the patient; and
a communication network linking the above components;
the health care facility server comprising:
communicating means for exchanging data with external equipment via the communication network;
database means for storing data regarding treatment methods for the patient and the status of treatment;
receiving means for receiving data from the medical institution regarding prescribed treatment methods for patients of the institution;
transmitting means for transmitting daily amounts of required exercise and energy per nutrient to be consumed, and other data required for treatment, to the portable terminal of the patient; and
recording means for receiving and recording data from the portable terminal of the patient regarding amounts of energy expended through exercise, amounts of energy per nutrient consumed, and other data related to treatment;
the food provision facility server comprising:
communicating means for exchanging data with external equipment via the communication network;
database means for storing data regarding energy per nutrient in foods provided by the food provision facility; and
searching and transmitting means for searching the database means in response to requests from the portable terminal and transmitting to that terminal data regarding the energy per nutrient in the requested foods;
the medical institution server comprising:
communicating means for exchanging data with external equipment via the communication network;
transmitting means for transmitting data regarding the treatment methods for the patient to the health care facility server; and
referencing means for referencing data regarding the status of treatment for the patient recorded in a database at the health care facility;
the portable terminal comprising:
communicating means for exchanging data with external equipment via the communication network;
receiving and displaying means for receiving and displaying daily amounts of required exercise and energy per nutrient to be consumed, and other data required for treatment, from the health care facility server;
inputting means for inputting data regarding the amounts of energy in consumed foods and the amounts of energy expended through exercise;
receiving means for receiving data from the food provision facility server regarding the energy per nutrient in foods about to be consumed at the food provision facility; and
transmitting means for transmitting data regarding the amounts of energy in consumed foods, the amounts of energy expended through exercise, and other data related to treatment, to the health care facility server.

8. A self-administered health support system for supporting a patient's self-administration of diet and exercise, comprising:
a server at a health care facility;
a server at a food provision facility;
a server at a medical institute;
a portable terminal carried by the patient; and
a communication network linking the above components;
the health care facility server comprising:
communicating means for exchanging data with external equipment via the communication network;
database means for storing personal data on the patient, data regarding treatment methods for the patient, data regarding the status of therapeutic exercise and diet regimens followed by the patient, data on medicines taken by the patient, and biological measurements of the patient;
receiving and storing means for receiving and storing data from the medical institution including personal data on the patient, prescribed treatment methods for the patients, and other data required for treatment;
transmitting means for transmitting daily amounts of required exercise and energy per nutrient to be consumed, dosage and administration of medicines, instructions for biological measurements, and other data required for treatment, to the portable terminal of the patient; and
recording means for receiving and recording data from the portable terminal of the patient regarding amounts of energy expended through exercise, amounts of energy per nutrient consumed, dosage and administration of medicines, execution of biological measurements, and other data related to treatment;
the food provision facility server comprising:
communicating means for exchanging data with external equipment via the communication network;
database means for storing data regarding energy per nutrient in foods provided by the food provision facility; and
searching and transmitting means for searching the database means in response to requests from the portable terminal and transmitting to that terminal data regarding the energy per nutrient in the requested foods;
the medical institution server comprising:
communicating means for exchanging data with external equipment via the communication network;
transmitting means for transmitting personal data on the patient, prescribed treatment methods for the patient, and other data required for treatment to the health care facility server; and
referencing means for referencing data regarding the status of therapeutic exercise and diet regimens followed by the patient, data on medicines taken by the patient, and biological measurements of the patient recorded in a database at the health care facility;
the portable terminal comprising:
communicating means for exchanging data with external equipment via the communication network;
receiving and displaying means for receiving and displaying daily amounts of required exercise and energy per nutrient to be consumed, dosage and administration of medicines, instructions for biological measurements, and other data required for treatment from the health care facility server;
inputting means for inputting data regarding the amounts of energy per nutrient in consumed foods;
inputting means for inputting the amounts of energy expended through exercise;
receiving means for receiving data from the food provision facility server regarding the energy per nutrient in foods about to be consumed at the food provision facility;
inputting means for inputting data from a motion measuring device that detects human movement and measures the amount of energy consumed through that movement;
inputting means for inputting data from a sphygmomanometer and other biological data measuring devices, and data from a treadmill and other auxiliary exercise equipment;
instructing means for prompting the patient to take medicines and measure biological data;
display means for displaying data regarding the amount of energy in foods consumed and the amount of energy expended through exercise; and
transmitting means for transmitting data regarding the amounts of energy in consumed foods, the amounts of energy expended through exercise, and biological data measurements to the health care facility server.

9. A self-administered health support system as recited in claim 7 or claim 8, wherein the health care facility server further comprises:
database means for storing data regarding the energy per nutrient in various foods; and
means for receiving data from the food provision facility server regarding foods provided at that facility, calculating the amounts of energy per nutrient in those foods based on data stored in the data base means, and returning the calculated data to the food provision facility server; and
the food provision facility server further comprises:
transmitting means for transmitting requests to calculate the energy per nutrients included in foods provided at the food provision facility to the server at the health care facility; and
database means for receiving and storing from the health care facility server calculated data on the energy per nutrients included in foods provided at the food provision facility.

10. A self-administered health support system as recited in claim 7 or claim 9, further comprising:
means for including names of foods and patterns or numbers to serve as an index for extracting data regarding energy per nutrient in the foods from the database in a menu of foods provided by the food provision facility in order to transmit such data from the server at the food provision facility to the patient's portable terminal;
wireless communication means for exchanging data wirelessly with the portable terminal; and
extracting and transmitting means for extracting data on energy per nutrient for foods stored in the database based on indexes transmitted wirelessly from the portable terminal and transmitting the extracted data to the portable terminal; wherein
the portable terminal further comprises:
wireless communication means for exchanging data wirelessly with the food provision facility terminal;
inputting means for inputting data from a device for reading patterns or numbers;
transmitting means for transmitting index data based on the patterns or numbers read from the menu to the food provision facility server wirelessly;
receiving means for receiving data regarding energy per nutrient in the foods from the food provision facility server; and
display means for determining and displaying the appropriateness of consuming the foods based on data regarding energy per nutrient in the selected foods and energy per nutrient to be consumed daily, as directed by the medical institution.

11. A self-administered health support system as recited in claim 10, wherein the patterns provided in the menus at the food provision facility are barcodes, and the device for reading the patterns is a barcode reader.

12. A self-administered health support system as recited in any of claims 7 through 10, wherein a portable telephone is used as the communicating means of the portable terminal for communicating with the communication network.

13. A self-administered health support system as recited in any of claims 7 through 12, wherein the food provision facility server and the portable terminal further comprise a wireless communication device as the transmitting and receiving means between the food provision facility server and the portable terminal.

14. A self-administered health support system as recited in any of claims 8 through 13, wherein an IC card or other portable storage medium is used as the inputting means for inputting data from the biological data measuring device into the portable terminal, and the portable terminal further comprises a device for reading from and writing to the portable storage medium.

15. A self-administered health support system as recited in claim 14, wherein some or all of the device for reading patterns and numbers, the wireless communication device for communicating with the food provision facility, the motion measuring device, and the device for reading from and writing to the portable storage medium are built into the portable terminal.

16. A self-administered health support system as recited in claim 14, wherein a portable telephone function is built into the portable terminal and some or all of the device for reading patterns and numbers, the wireless communication device for communicating with the food provision facility, the motion measuring device, and the device for reading from and writing to the portable storage medium are built into the portable terminal.

17. A self-administered health support system as recited in claim 7 or claim 8, wherein a plurality of health care facilities are provided according to the number of users or the size of a controlling region, with the present system being managed by each health care facility, and the servers of each health care facility are interconnected via a communication network.

18. A self-administered health support system as recited in any of claims 7 through 17, wherein the communications network is the Internet.

19. A self-administered health support system as recited in claim 18, wherein the server at the health care facility is a web server comprising:
means for presenting a web page on the Internet;
means for providing data to the user via the web page regarding guidance in using the system and treatment;
means for answering questions from users via the web page;
means for providing counseling to users via the web page in order to alleviate treatment concerns and emotional distress;
means for exchanging opinions with other users via the web page; and
means for providing via electronic mail data regarding the user's self-administered status to a health insurance institution that is engaged in a contract with the user after receiving consent from the user.
